(19) United States
(12) Patent Application Publication
(10) Pub. No.: US 2024/0398614 A1
(43) Pub. Date: Dec. 5, 2024

(54) COOLING DEVICES WITH FLEXIBLE SENSORS

(71) Applicant: Zeltiq Aesthetics, Inc., North Chicago, IL (US)

(72) Inventors: Mitchell Levinson, Pleasanton, CA (US); Jesse N. Rosen, Albany, CA (US)

(21) Appl. No.: 18/629,120

(22) Filed: Apr. 8, 2024

Related U.S. Application Data
(63) Continuation of application No. 16/824,530, filed on Mar. 19, 2020, now Pat. No. 11,986,421.
Application No. 16/824,530 is a continuation of application No. 11/528,189, filed on Sep. 26, 2006, now abandoned.

(51) Int. Cl. A61F 7/10; A61B 5/00; A61B 17/00; A61B 18/00; A61B 90/00; A61F 7/00; A61F 7/02; G05D 23/19; G05D 23/22; G05D 23/24

(52) U.S. Cl. CPC A61F 7/10; A61B 5/411; A61B 5/6843; G05D 23/1919; G05D 23/22; G05D 23/24; A61B 2017/00088; A61B 2017/00092; A61B 2017/00101; A61B 2018/00011; A61B 2018/00023; A61B 2018/00047; A61B 2018/00744; A61B 2018/00791; A61B 2090/064; A61B 2562/164; A61F 2007/0001; A61F 2007/0075; A61F 2007/0228; A61F 2007/029

(57) ABSTRACT

A device for exchanging heat with a subject having skin is provided. The device includes a heat exchanging member having a heat transfer surface configured to form a heat conducting interface with the subject's skin. The device further includes a substantially flexible sensing device disposed in the interface between the heat exchanging member and the subject's skin. The sensing device is configured to measure a parameter of the interface without substantially impeding heat transfer between the heat exchanging member and the subject's skin.

### TECHNICAL FIELD

The present application relates to cooling devices, systems, and methods for exchanging heat with subcutaneous lipid-rich cells of a subject.

### BACKGROUND

As statistics have shown, excess body fat increases the likelihood of developing various diseases and can detract from personal appearance and athletic performance. One conventional technique of controlling excess body fat is liposuction that can selectively remove body fat to sculpt a person's body. One drawback of liposuction is that it is a complex surgical procedure that can have serious and occasionally even fatal complications.

Conventional non-invasive treatments for removing excess body fat typically include topical agents, weight-loss drugs, regular exercise, dieting, or a combination of these treatments. One drawback of these treatments is that they may not be effective or even possible under certain circumstances. For example, when a person is physically injured or ill, regular exercise may not be an option. Similarly, weight-loss drugs or topical agents are not an option when they cause an allergic or negative reaction.

Other non-invasive treatment methods include applying heat to a zone of subcutaneous lipid-rich cells. U.S. Pat. No. 5,948,011 discloses altering subcutaneous body fat and/or collagen by heating the subcutaneous fat layer with radiant energy while cooling the surface of the skin. Another promising method of reducing subcutaneous fat cells is to cool the target cells as disclosed in U.S. Patent Publication No. 2003/0220674,
the entire disclosure of which is incorporated herein. U.S. Patent Publication No.
2003/0220674 also discloses methods for selective removal of lipid-rich cells, and avoidance of damage to other structures including dermal and epidermal cells.

In any of these non-invasive treatment methods, temperatures at heat transfer interfaces (e.g., between a treatment device and a skin surface) are important for safety reasons. High interface temperatures may cause scorching of the skin surface, and low interface temperatures may cause frostbite. Therefore, effective devices and methods for accurately measuring the interface temperatures would be desirable.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn are not intended to convey any information regarding the actual shape of the particular elements, and have been solely selected for ease of recognition in the drawings.

FIG. 1 is an isometric view of a system for exchanging heat with a subject in accordance with an embodiment of the invention.

FIG. 2 is a partially exploded isometric view of a cooling device with flexible sensors in accordance with an embodiment of the invention.

FIG. 3 is a partially exploded isometric view of a cooling device with flexible sensors in accordance with another embodiment of the invention.

FIG. 4 is a front view of a flexible sensing device in accordance with another embodiment of the invention.

FIG. 5 is a back view of a flexible sensing device in accordance with another embodiment of the invention.

FIG. 6 is a front view of a flexible sensing device in accordance with a further embodiment of the invention.

FIG. 7 is a block diagram showing computing system software modules for exchanging heat with a subject in accordance with an embodiment of the invention.

### DETAILED DESCRIPTION

### A. Overview

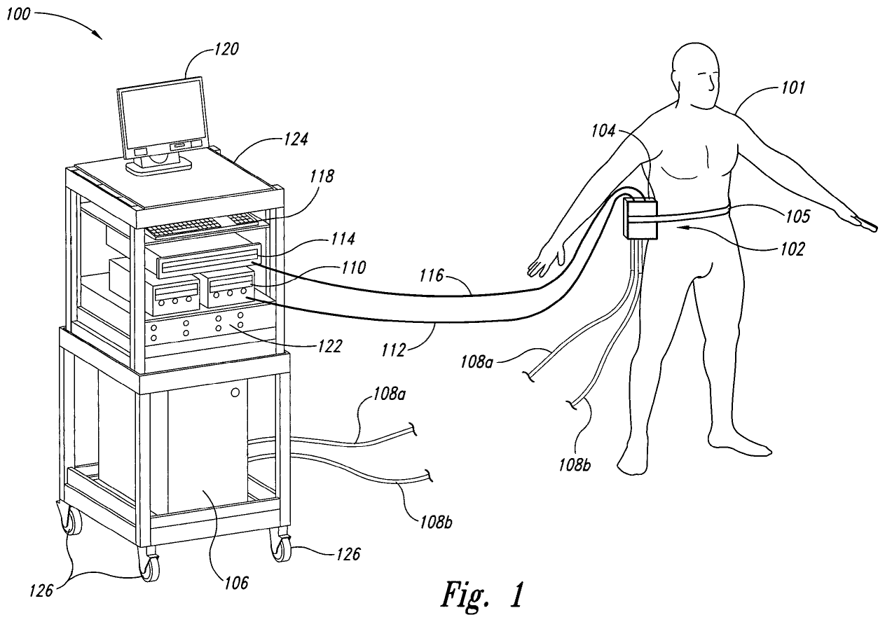

The present disclosure describes devices, systems, and methods for exchanging heat with subcutaneous lipid-rich cells. The term “subcutaneous tissue” means tissue lying underneath the dermis and includes adipocytes (fat cells) and subcutaneous fat. It will be appreciated that several of the details set forth below are provided to describe the following embodiments in a manner sufficient to enable a person skilled in the relevant art to make and use the disclosed embodiments. Several of the details and advantages described below, however, may not be necessary to practice certain embodiments of the invention. Additionally, the invention can include other embodiments that are within the scope of the claims but are not described in detail with respect to FIGS. 1-7.

Reference throughout this specification to “one embodiment” or “an embodiment” means that a particular feature, structure, or characteristic described in connection with the embodiment is included in at least one embodiment of the present invention. Thus, the occurrences of the phrases “in one embodiment” or “in an embodiment” in various places throughout this specification are not necessarily all referring to the same embodiment. Furthermore, the particular features, structures, or characteristics may be combined in any suitable manner in one or more embodiments.

The headings provided herein are for convenience only and do not interpret the scope or meaning of the claimed invention.

One aspect is directed toward a cooling device for removing heat from subcutaneous lipid-rich cells of a subject's skin. The cooling device can include a heat exchanging member having a heat transfer surface configured to form a heat conducting interface with the subject's skin to remove heat from the lipid-rich cells such that the lipid-rich cells are affected while non-lipid-rich cells in the epidermis are not affected. The cooling device can further include a substantially flexible sensing device disposed in the interface between the heat exchanging member and the subject's skin, wherein the sensing device is configured to sense a parameter at the interface without substantially impeding heat transfer between the heat exchanging member and the subject's skin.

Another aspect is directed toward a sensing device for measuring parameters of a heat transfer interface between a subject having skin and a cooling device having a substantially flexible substrate positioned in the heat transfer interface between the subject's skin and the cooling device. The substrate can include a temperature sensor disposed on the surface of the substrate. According to aspects, the temperature sensor is configured to measure a temperature of the heat transfer interface without substantially impeding heat transfer between the cooling device and the subject's skin.

Another aspect is directed toward a method of applying a cooling device configured for removing heat from a subject having skin, the method including disposing a sensing device proximate to the cooling device, the sensing device being substantially flexible and at least partially conforming to the cooling device. The method can further include positioning the cooling device and the sensing device proximate to the subject's skin, wherein the cooling device and the subject's skin form a heat transfer interface, in which the sensing device is positioned. The method can further include measuring a parameter of the heat transfer interface using the sensing device and removing heat from a region of the subject's skin under the epidermis such that lipid-rich cells are affected while non-lipid-rich cells in the epidermis are not affected.

### B. System for Selectively Reducing Lipid-Rich Cells

FIG. 1 is an isometric view of a system 100 for exchanging heat from subcutaneous lipid-rich cells of a subject 101 in accordance with an embodiment of the invention. The system 100 can include a cooling device 104 placed at an abdominal area 102 of the subject 101 or another suitable area for exchanging heat from the subcutaneous lipid-rich cells of the subject 101. The cooling device 104 can be fastened to the subject 101 using, for example, a mechanical fastener (e.g., a belt 105), an adhesive (e.g., an epoxy), suction (e.g., a vacuum or reduced pressure) or any other mechanisms. The cooling device 104 can be configured to heat and/or cool the subject

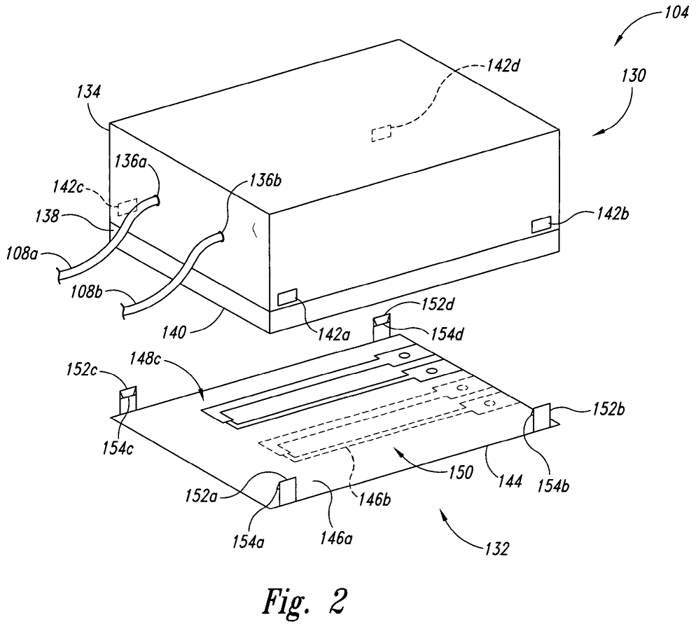

101. Various embodiments of the cooling device 104 are described in more detail below with reference to FIGS. 2-7.

In one embodiment, the cooling device 104 is configured to cool subcutaneous lipid-rich cells of the subject 101. In such cases, the system 100 can further include a cooling unit 106 and fluid lines 108a-b connecting the cooling device 104 to the cooling unit 106. The cooling unit 106 can remove heat from a coolant to a heat sink and provide the chilled coolant to the cooling device 104 via the fluid lines 108a-b. Examples of the circulating coolant include water, glycol, synthetic heat transfer fluid, oil, a refrigerant, and any other suitable heat conducting fluids. The fluid lines 108a-b can be hoses or other conduits constructed from polyethylene, polyvinyl chloride, polyurethane, and other materials that can accommodate the particular circulating coolant. The cooling unit 106 can be a refrigeration unit, a cooling tower, a thermoelectric chiller, or any other device capable of removing heat from a coolant or municipal water supply.

The cooling device 104 can also include one or more thermoelectric elements, such as Peltier-type thermoelectric elements. In such cases, the system 100 can further include a power supply 110 and a processing unit 114 operatively coupled to the cooling device 104 via electrical cables 112, 116. In one embodiment, the power supply 110 can provide a direct current voltage to the cooling device 104 to effectuate a heat removal rate from the subject 101. The processing unit 114 can monitor process parameters via sensors (not shown in FIG. 1) placed proximate to the cooling device 104 and adjust the heat removal rate based on the process parameters. The processing unit 114 can include any processor, Programmable Logic Controller, Distributed Control System, and the like.

The processing unit 114 can be in electrical communication with an input device 118, an output device 120, and/or a control panel 122. The input device 118 can include a keyboard, a mouse, a touch screen, a push button, a switch, a potentiometer, and any other device suitable for accepting user input. The output device 120 can include a display screen, a printer, a medium reader, an audio device, and any other device suitable for providing user feedback. The control panel 122 can include indicator lights, numerical displays, and audio devices. In the embodiment shown in FIG. 1, the processing unit 114, power supply 110, control panel 122, cooling unit 106, input device 118, and output device 120 are carried by a rack 124 with wheels 126 for portability. In another embodiment, the various components can be fixedly installed at a treatment site.

One expected advantage of the system 100 is that the cooling device 104 can be applied to the subject 101 irrespective of the current physical condition of the subject 101. For example, the system 100 can be applied even when the subject 101 is not ambulatory or is ill. Another expected advantage is that the system 100 can remove or affect fat non-invasively without piercing the skin of the subject 101. Yet another expected advantage is that the system 100 is compact and can be used in an outpatient facility or a doctor's office. A further expected advantage is that the system 100 can quickly cool lipid-rich cells in a subcutaneous layer without requiring high-voltage power supplies.

### C. Cooling Devices With Flexible Sensors

FIG. 2 is a partially exploded isometric view of a cooling device 104 in accordance with one embodiment of the invention and suitable for use in the system 100. In this example, the cooling device 104 includes a heat exchanging member 130 and a sensing device 132 affixed to the heat exchanging member 130. The cooling device 104 is generally configured for manual positioning, and/or it can be strapped or otherwise configured to be releasably attached to the subject 101. The sensing device 132 is configured to measure a parameter at an interface of the cooling device 104 and the skin of the subject 101.

The heat exchanging member 130 can include a housing 134 and fluid ports 136a-b coupled to the fluid lines 108a-b. In one example, the housing 134 is generally rectangular, but in other examples, the housing 134 can be cubic, spherical, semi-spherical, or any other desired shape. The housing 134 can include features for attaching the sensing device 132. In the illustrated example, the housing 134 includes a plurality of indentations 142 (identified individually as 142a-d). In other examples, the housing 134 can include threaded apertures, channels, slots, pegs, or any other suitable attachment mechanism. The housing 134 can be constructed from polymeric materials, metals, ceramics, woods, and/or other suitable materials.

The heat exchanging member 130 can further include an interface member 138 at least partially in the housing 134. The interface member 138 has a heat exchanging surface 140 for transferring heat to/from the subject 101. In one example, the heat exchanging surface 140 is generally planar, but in other examples, the heat exchanging surface 140 can be non-planar (e.g., curved, faceted, etc.) The interface member 138 can be constructed from any suitable material with a thermal conductivity greater than 0.05 Watts/Meter Kelvin, and in many examples, the thermal conductivity is more than 0.1 Watts/Meter Kelvin. Examples of suitable materials include aluminum, copper, other metals, metal alloys, graphite, ceramics, some polymeric materials, composites, or fluids contained in a flexible membrane. In other embodiments, portions of the heat exchanging surface 140 can be constructed from an insulating material with a thermal conductivity less than 0.05 Watts/Meter Kelvin.

The sensing device 132 can include a substrate 144 having a first surface 146a and a second surface 146b. The substrate 144 can have a profile generally corresponding to the profile of the interface member 138. For example, in the illustrated example, the substrate 144 is a flat and generally rectangular film that generally matches the profile of the illustrated heat exchanging surface 140 of the interface member 138. In other examples, the substrate can have curved, faceted, or other desired profiles to correspond to the interface member 138. In further examples, the substrate 144 can have a profile that corresponds to only a portion of the interface member 138.

The substrate 144 can be substantially flexible to conform to the interface member 138 and have sufficient heat conductivity. As a result, the sensing device 132 does not substantially impede heat transfer between the cooling device 104 and the subject 101. In one example, the substrate 144 can be a thin film constructed from polyimide, polyamide, polycarbonate, or any other suitable material with sufficient heat conductivity. In another example, the substrate 144 can be a thick film attached to a backing material (not shown, e.g., paper, plastic, etc.) with an adhesive. According to aspects of the invention, the substrate 144 can be peeled off the backing material and adhered to the interface member 138 during assembly.

The substrate 144 can also include attachment features for affixing the sensing device 132 to the housing 134. In the illustrated example, the substrate 144 includes clips 152 (identified individually as 152a-d) that correspond to the indentations 142 of the housing 134. Individual clips 152 include protrusions 154 (identified individually as 154a-d) that can fit inside the indentations 142. During assembly, the substrate 144 is snapped onto the housing 134 with the first surface 146a facing the interface member 138. The clips 152 fasten the substrate 144 onto the housing 134 when the protrusions 154 of the clips 152 engage the indentations 142. In other examples, the substrate 144 can be attached to the housing 134 using screws, pins, hinges, or any other suitable attachment mechanism.

The sensing device 132 can also include at least one sensor disposed on the first and/or second surfaces 146a-b of the substrate 144 to measure a parameter of the interface. In the illustrated example, the sensing device 132 includes a first temperature sensor 148 disposed on the first surface 146a and a second temperature sensor 150 disposed on the second surface 146b. The first temperature sensor 148 contacts the interface member 138 after assembly to directly measure temperatures of the heat exchanging surface 140. The second temperature sensor 150 contacts the subject's skin to directly measure skin temperatures during use. In other examples, the sensing device 132 can include other types of sensors or a greater or smaller number of sensors disposed on the substrate 144. For example, the substrate 144 can include only one temperature sensor disposed on the second surface 146b for measuring the skin temperatures or multiple temperature sensors on the second surface 146b for redundancy. Alternatively, or in conjunction with multiple sensors, the substrate can include pressure sensors, transmissivity sensors, bioresistance sensors, ultrasound sensors, optical sensors, infrared sensors, heat flux, any other desired sensors, or any combination thereof.

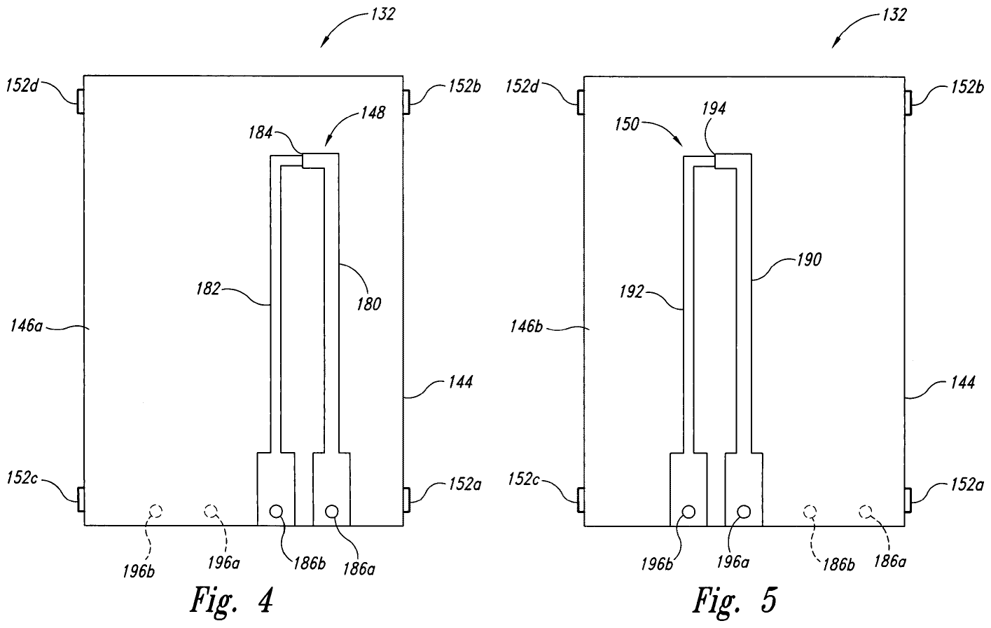

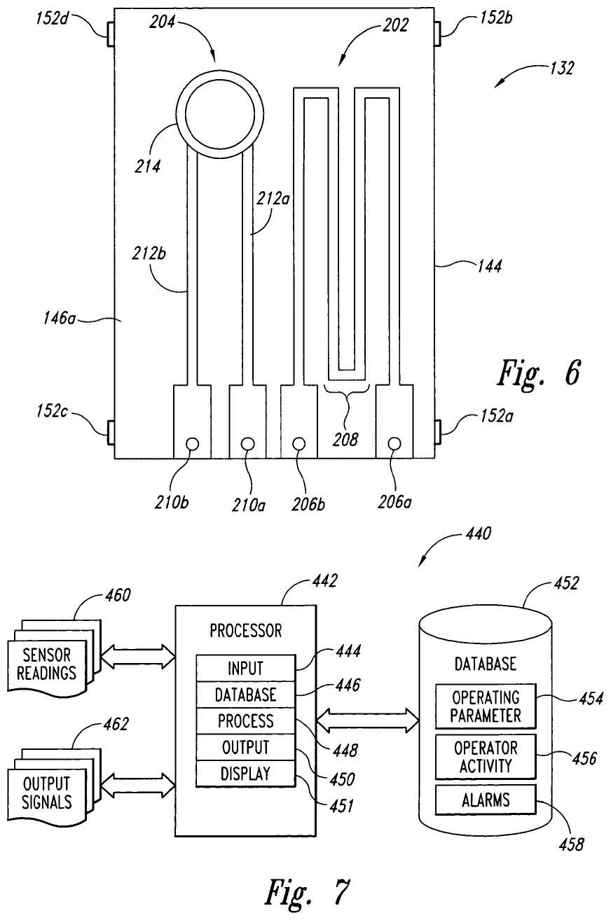

In the illustrated example, the first and second temperature sensors 148, 150 are configured as thermocouples as described in more detail below with reference to FIGS. 4 and 5. In other examples, the temperature sensors 148, 150 can be configured as Resistive Temperature Detectors (RTD), thermistors, thermopiles, or other types of temperature sensors as described in more detail below with reference to FIG. 6. A thermopile is essentially a series of thermocouples and can be wired to measure temperature difference between two surfaces. In one embodiment, the thermocouples are laminated onto a Kapton backing and measure the temperature across the Kapton backing very accurately, which can then be converted to heat flux. In a further example, the sensing device 132 can include pressure sensors, transmissivity sensors, bioresistance sensors, ultrasound sensors, optical sensors, infrared sensors, heat flux, or any other desired sensors.

A coupling agent may be applied to the subject's skin or to the interface member 138 to provide improved thermal conductivity. The coupling agent may include polypropylene glycol, polyethylene glycol, propylene glycol, and/or glycol. Glycols, glycerols, and other deicing chemicals are efficient freezing-point depressants and act as a solute to lower the freezing point of the coupling agent. Propylene glycol (CH3CHOHCH2OH) is one exemplary component of deicer or non-freezing coupling agents. Other components include polypropylene glycol (PPG), polyethylene glycol (PEG), polyglycols, glycols, ethylene glycol, dimethyl sulfoxide, polyvinyl pyridine, calcium magnesium acetate, sodium acetate, and/or sodium formate. The coupling agent preferably has a freezing point in the range of −40° C. to 0° C., more preferably below −10° C. as further described in U.S. Provisional Application 60/795,799, entitled Coupling Agent For Use With a Cooling Device For Improved Removal of Heat From Subcutaneous Lipid-Rich Cells, filed on Apr. 28, 2006, herein incorporated in its entirety by reference.

In operation, an operator can place the cooling device 104 proximate to the subject's skin to form a heat exchanging interface. In one embodiment, the operator can press the cooling device 104 against the subject's skin. In another embodiment, the operator can clamp a portion of the subject's skin between the cooling device 104 and another device, such as a device similar in function and structure to the cooling device 104. The operator can then exchange heat with the subject's skin using the cooling device 104. In one embodiment, the operator can cool the subject's subcutaneous tissues by circulating a coolant through the heat exchanging member 130 via the fluid lines 108a-b. Heat can then be removed from the subject's skin, past the sensing device 132, to the heat exchanging member 130. By cooling the subcutaneous tissues to a temperature lower than 37° C., preferably between about −20° C. to about 20° C., more preferably between about −20° C. to about 10° C., more preferably between about −15° C. to about 5° C., more preferably between about −10° C. to about 0° C., subcutaneous lipid-rich cells can be selectively affected. The affected cells are resorbed into the subject through natural processes. In any of these embodiments, the operator can monitor and control the heat exchanging process by measuring skin and interface temperatures using the sensing device 132. In one example, the operator can prevent excessively cooling the subject's skin by maintaining the skin and/or interface temperatures at a safe level. In other examples, the skin and/or the interface temperatures can be used as process variables to automatically control the heat exchanging process.

One expected advantage of using the cooling device 104 is the reduced risk of overcooling the subject's skin because the heat transfer interface temperature can be directly measured. As is known, heat conduction through an object creates a temperature gradient along a heat transfer path. For example, the temperature of the subject's dermis can be higher than that of the subject's epidermis during heat conduction. As a result, if the dermis temperature or a temperature internal to the cooling device, is used to control a cooling process, the epidermis temperature may be too high or too low. Consequently, using directly measured interface temperatures (e.g., at the epidermis) can reduce the risk of overheating or overcooling the subject's skin.

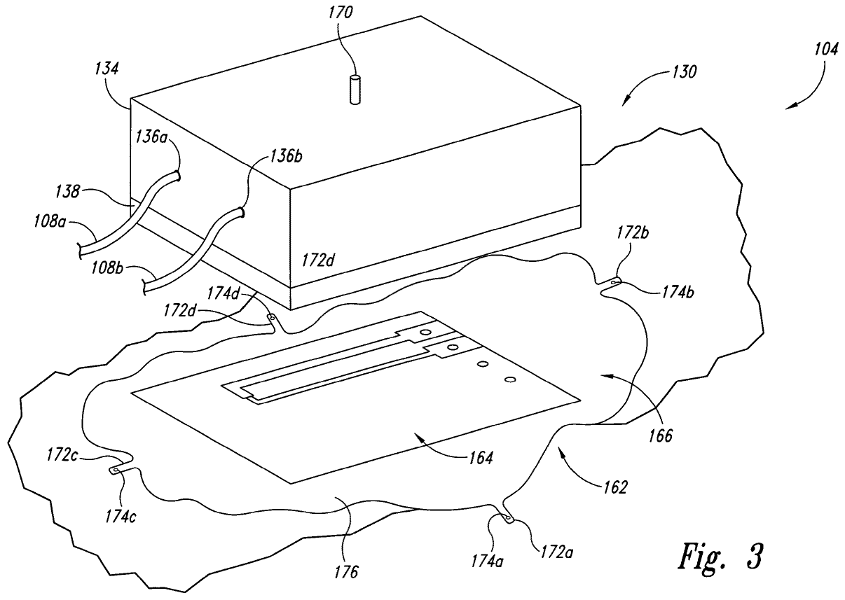

The cooling device 104 can have many additional embodiments with different and/or additional features without detracting from the operation of the cooling device 104. For example, the cooling device 104 can be configured to be a handheld device as described in U.S. patent application Ser. No. 11/359,092 entitled Cooling Device For Removing Heat From Subcutaneous Lipid-Rich Cells, the entire disclosure of which is herein incorporated by reference. The heat exchanging member 130 can include thermoelectric heat exchanging members (e.g., Peltier-type elements), cryogenic elements (e.g., liquid Nitrogen evaporator), or other types of suitable heat exchanging elements. For example, the cooling device 104 can be configured as a plurality of thermoelectric heat exchanging members contained on a hinged frame to allow rotation about at least one axis as described in U.S. Patent Application entitled Cooling Device Having a Plurality of Controllable Thermoelectric Cooling Elements to Provide a Predetermined Cooling Profile filed concurrently herewith, application number not yet assigned, the entire disclosure of which is herein incorporated by reference. The sensing device 132 can also be incorporated into a sleeve that can isolate the subject 101 from the heat exchanging member 130 as described below in more detail with reference to FIG. 3.

### D. Cooling Devices With Sleeve Sensors

FIG. 3 is an alternative example of the cooling device 104 in accordance with one example of the invention for use in the system 100. This alternative example, and those alternative examples, and other alternatives described herein, is substantially similar to previously described examples, and common acts and structures are identified by the same reference numbers. Only significant differences in operation and structure are described below. In this example, the cooling device 104 includes a sleeve 162 having a first sleeve portion 164 and a second sleeve portion 166. The first sleeve portion 164 can be generally similar in structure and function to the sensing device 132 of FIG. 2. The second sleeve portion 166 can be an isolation layer extending from the first sleeve portion 164. For example, the second sleeve portion 166 can be constructed from latex, rubber, nylon, Kevlar®, or other substantially impermeable or semipermeable material. The second sleeve portion 166 can prevent any contact between the subject's skin and the heat exchanging member 130. In one example, the sleeve 162 can be reusable. In other examples, the sleeve 162 can be disposable. The sleeve 162 may be provided sterile or non-sterile.

The second sleeve portion 166 can also include attachment features to affix the sleeve 162 to the housing 134. In the illustrated example, the second sleeve portion 166 includes four brackets 172 (identified individually as 172a-d), each located at a corner of the second sleeve portion 166. Individual brackets 172 include an aperture 174 (identified individually as 174a-d) that corresponds to an attachment point 170 of the housing 134. During assembly, the apertures 174 of the brackets 172 can fit over the attachment point 170 such that the second sleeve portion 166 at least partially encloses the heat exchanging member 130. In another example, the second sleeve portion 166 can include brackets that can engage each other. For example, the bracket 172a can include a pin that can engage the aperture 174d of the bracket 172d. During assembly, the second sleeve portion 166 can wrap around the housing 134 and be held in place by engaging the brackets 172 with each other. In a further example, the second sleeve portion 166 can include a flexible member (not shown, e.g., an elastic band) at an outer edge 176 of the second sleeve portion 166 that can hold the sleeve 162 over the housing 134 during assembly. In a further example, the second sleeve portion 166 can include a releasable attachment member (not shown, e.g., Velcro® or snaps) at the outer edge 176 of the second sleeve portion 166 that can hold the sleeve 162 over the housing 134 during assembly. In yet another example, adhesive can hold the second sleeve portion 166 to the housing 134.

In addition to the expected advantages described above, one expected advantage of using the sleeve 162 is the improved sanitation of using the cooling device 104. The sleeve 162 can prevent cross-contamination between the subject's skin and the heat exchanging member 130 because the sleeve 162 is substantially impermeable. Also, operating expense of the cooling device 104 can be reduced because the heat exchanging member 130 does not need to be sanitized after each use.

The sleeve 162 can have many additional embodiments with different and/or additional features without detracting from its operation. For example, the first and second sleeve portions 164, 166 can be constructed from the same material (e.g., polyimide) or different materials. The sleeve 162 can include an adhesive layer (not shown) that binds the sleeve 162 to the housing 134. Alternatively, a coupling gel (not shown) can be applied between the sleeve 162 and the interface member 138.

### E. Sensing Devices

FIG. 4 is a front view and FIG. 5 is a back view of the sensing device 132 illustrating several features in more detail. The first temperature sensor 148 can include a first metal trace 180 and a second metal trace 182 spaced apart from the first metal trace 180. The first metal trace 180 includes a first terminal portion 186a, and the second metal trace 182 includes a second terminal portion 186b. The first and second metal traces 180,182 join at one end to form a bi-metal junction 184. In the illustrated embodiment, the first and second metal traces 180, 182 are generally parallel to each other. In other examples, the first and second metal traces 180, 182 can be at an angle.

The first and second metal traces 180, 182 can be disposed onto the substrate 144 using techniques including, for example, bonding, laminating, sputtering, etching, printing, or other suitable methods. The first and second metal traces 180, 182 can include iron, constantan, copper, nicrosil, platinum, rhodium, tungsten, or other suitable metals or metal alloys. The first and second metal traces 180, 182 can form thermocouples of the types J, K, T, E, N, R, S, U, B, and other desired types.

In the illustrated example, the second temperature sensor 150 is generally similar in structure and function to the first temperature sensor 148. For example, the second temperature sensor 150 can include metal traces 190, 192 joined at an end to form a bi-metal junction 194 and terminal portions 196a-b. In one embodiment, the first and second temperature sensors 148, 150 can be the same type (e.g., type T). In another embodiment, the first and second temperature sensors 148, 150 can be of different types.

FIG. 6 is an alternative example of the sensing device 132 in accordance with one example of the invention for use in the system 100. In this example, the sensing device 132 includes an RTD 202 and a pressure sensor 204 disposed on the first surface 146a of the substrate 144. The RTD 202 includes a first RTD terminal 206a, a second RTD terminal 206b, and a resistance portion 208 between the two RTD terminals 206a-b. The resistance portion 208 can be constructed from platinum, gold, silver, copper, nickel or a combination of metals, such as nickel-iron, or any other materials or combinations of materials with sufficient temperature resistance change. A preferred embodiment includes a nickel-iron metal foil. The pressure sensor 204 includes terminals 210a-b, a pressure sensing portion 214, and leads 212a-b connecting the terminals 210a-b to the pressure sensing portion 214. The pressure sensor 204 can be generally similar to a FlexiForce® load sensor (Model No. A201) manufactured by Tekscan, Inc. of South Boston, Massachusetts.

In operation, the RTD 202 senses the interface temperature between the interface member 138 (FIG. 2) and the subject's skin, and the pressure sensor 204 senses the pressure applied to the subject's skin from the cooling device 104 (FIGS. 2 and 3). An operator can then adjust the pressure applied to the subject's skin and/or the heat exchanging rate based on these measurements.

One expected advantage of using the sensing device 132 is the improved uniformity of heat transfer across the heat exchanging interface. If the contact between the interface member 138 and the subject's skin is poor, air gaps in the interface can substantially impede the heat transfer between the cooling device 104 and the subject's skin and cause faulty interface temperature measurements. By using the sensing device 132, the operator can monitor and correct the amount of pressure applied to the subject's skin to ensure good contact at the heat exchanging interface. Consequently, uniformity of the heat transfer across the interface can be improved.

### F. Computing System Software Modules

FIG. 7 illustrates a functional block diagram showing exemplary software modules 440 suitable for use in the processing unit 114 (FIG. 1). Each component can be a computer program, procedure, or process written as source code in a conventional programming language, such as the C++ programming language, and can be presented for execution by the CPU of a processor 442. The various implementations of the source code and object and byte codes can be stored on a computer-readable storage medium or embodied on a transmission medium in a carrier wave. The modules of the processor 442 can include an input module 444, a database module 446, a process module 448, an output module 450, and optionally, a display module 451. In another embodiment, the software modules 440 can be presented for execution by the CPU of a network server in a distributed computing scheme.

In operation, the input module 444 accepts an operator input, such as process setpoint and control selections, and communicates the accepted information or selections to other components for further processing. The database module 446 organizes records, including operating parameter 454, operator activity 456, alarm 458, and facilitates storing and retrieving of these records to and from a database 452. Any type of database organization can be utilized, including a flat file system, hierarchical database, relational database, or distributed database, such as provided by Oracle Corporation, Redwood Shores, California.

The process module 448 generates control variables based on sensor readings 460 obtained from the sensing device 132 (FIG. 2), and the output module 450 generates output signals 462 based on the control variables. For example, the output module 450 can convert the generated control variables from the process module 448 into 4-20 mA output signals 462 suitable for a direct current voltage modulator. The processor 442 optionally can include the display module 451 for displaying, printing, or downloading the sensor readings 460 and output signals 462 via devices such as the output device 120 (not shown). A suitable display module 451 can be a video driver that enables the processor 442 to display the sensor readings 460 on the output device 120.

Throughout the description and the claims, the words “comprise,” “comprising,” and the like, unless the context clearly requires otherwise, are to be construed in an inclusive sense as opposed to an exclusive or exhaustive sense; that is to say, in a sense of “including, but not limited to.” Words using the singular or plural number also include the plural or singular number, respectively. When the claims use the word “or” in reference to a list of two or more items, that word covers all of the following interpretations of the word: any of the items in the list, all of the items in the list, and any combination of the items in the list.

The above detailed descriptions of embodiments of the invention are not intended to be exhaustive or limit the invention to the precise form disclosed above. While specific embodiments of, and examples for, the invention are described above for illustrative purposes, various equivalent modifications are possible within the scope of the invention, as those skilled in the relevant art will recognize. For example, while steps are presented in a given order, alternative embodiments may perform steps in a different order. The various embodiments described herein can be combined to provide further embodiments.

In general, the terms used in the following claims should not be construed to limit the invention to the specific embodiments disclosed in the specification, unless the above detailed description explicitly defines such terms. While certain aspects of the invention are presented below in certain claim forms, the inventors contemplate the various aspects of the invention in any number of claim forms. Accordingly, the inventors reserve the right to add additional claims after filing the application to pursue such additional claim forms for other aspects of the invention.

1. A device for removing heat from subcutaneous lipid-rich cells of a subject having skin, comprising:
i. a heat exchanging member having a heat transfer surface configured to form a heat conducting interface with the subject's skin to remove heat from the lipid-rich cells such that the lipid-rich cells are substantially affected while non-lipid-rich cells in the epidermis are not substantially affected; and
ii. a substantially flexible sensing device disposed in the interface between the heat exchanging member and the subject's skin, wherein the sensing device is configured to sense a parameter at the interface.

2.-30. (canceled)
